(19) United States
(12) Patent Application Publication
(10) Pub. No.: US 2022/0322970 A1
(43) Pub. Date: Oct. 13, 2022

(54) INNOVATIVE KIT THAT INCLUDES A WEARABLE FOR DETECTING, CHARACTERIZING, AND MONITORING INVOLUNTARY MOVEMENT AND ATTACHABLE NON-INTRUSIVE INTERVENTIONS TO RELIEVE TREMORS IN HUMAN LIMBS

(71) Applicants: Jagdish Singh, Mesa, AZ (US); Prasenjit Mandal, Pune (IN); Natasha Singh-Miller, Urbana, MD (US); Poonam Sharma, Wilsonville, OR (US)

(72) Inventors: Jagdish Singh, Mesa, AZ (US); Prasenjit Mandal, Pune (IN); Natasha Singh-Miller, Urbana, MD (US); Poonam Sharma, Wilsonville, OR (US)

(21) Appl. No.: 17/715,052

(22) Filed: Apr. 7, 2022

Related U.S. Application Data
(60) Provisional application No. 63/171,978, filed on Apr. 7, 2021.

(51) Int. Cl. A61B 5/11; A61B 5/00

(52) U.S. Cl. CPC A61B 5/1101; A61B 5/681; A61B 5/7455; A61B 5/7475; A61B 5/742; A61B 5/4082; A61B 5/7282; A61N 1/36031

(57) ABSTRACT

The present invention comprises a novel kit with a wearable unit along with associated intelligent entities and non-intrusive tremor relieving interventions. The interventions include an automatically controllable TENS intervention unit with associated cables and electrodes and a buzzer. The wearable unit along with associated intelligent entities detects involuntary movement signals (tremors) in the limbs by measuring the acceleration in multiple dimensions, and ingeniously analyzes the accelerations to identify and isolate occurrences of tremors. This information is analyzed and monitored over time to provide significant insight about the tremors such as the dominant frequency (or frequencies), percent time with tremor, median tremor duration, and intensity. The TENS unit provides electrical stimulation which can dampen the intensity of involuntary movement and/or temporarily suspend tremor activity. This (TENS) unit can be manually or automatically controlled. The automatic control mode controls the TENS unit depending on the onset of tremors, tremor duration, and intensity of tremors. Furthermore, the parameters of this automatic control can be adjusted.

### BACKGROUND OF THE INVENTION

### Field of Invention Statement

The present invention relates to novel wearable devices and companion software services to detect, characterize, and monitor involuntary movement caused by motion/tremor disorders (e.g., Parkinson's Disease, essential tremor, etc.). In addition, the wearable device kits include attachable non-intrusive interventions that include a transcutaneous electrical nerve stimulation (TENS) unit and a stimulating buzzer that can be used to supplement medications by further mitigating involuntary movement.

### Background

Millions of Americans have tremor disorders which involve involuntary movement of one or more parts of the body, can severely deteriorate lifestyle, and progressively incapacitate. For example, a 2014 study estimated 7 million Americans have essential tremors¹, and the Parkinson's Foundation estimates 1 million Americans have Parkinson's Disease². To date, there is no general “cure”, but many medications (often intolerable) can relieve symptoms and clinicians must adjust therapeutic plans with limited subjective resources.

In addition to medication, a variety of lifestyle aids do exist in the market. These products include numerous specialized pens and utensils to assist daily activities such as writing and eating. Also, multiple wearable gloves are available that attempt to stabilize wrist/forearm motions. The underlying technology for these gloves involves some sort of heavy weight or counter force to dampen motions.^{3,4} For example, the Weighted Hand Writing Glove contains “lead free steel shot” to help steady the hand when writing.³ Two gloves that have more advanced mechanical technology are the Tremelo glove targeted for those with essential tremors⁴ and the Steadi-One glove which contains a “smart fluid” to counteract involuntary movement⁵. The Steadi-One glove dampens tremors that occur above a certain frequency, while allowing for voluntary movement (e.g., such as writing) which occurs at lower frequencies.⁵ The Readi-Steadi glove includes sensors that detect tremor patterns and use this information to counter involuntary motions.⁶

To determine an appropriate treatment plan, establishing diagnosis is important. Yet this process often is time-consuming, expensive, and convoluted, where misdiagnosis can result. An article by Schrag et. al showed that out of 50 patients who were initially diagnosed with essential tremor, 50% of those were incorrect diagnoses after further study.⁷ Jain et. al showed that out of 71 patients, 37% were misdiagnosed with essential tremor.⁸ A recent article by The American Journal of Managed Care (January 2020) cited a poll that revealed approximately a quarter of more than 2000 participants with Parkinson's had been misdiagnosed.⁹ Moreover, of the individuals who were misdiagnosed, 48% were prescribed medication for a diagnosis they didn't have and 34% had worsening health.⁹ Furthermore, monitoring care of individuals over time and how they respond to treatment mostly involves subjective assessment with little or no quantitative or scientific corroboration.

Therefore, there is a need in the field of tremor disorders to provide more robust medical technologies to characterize tremors with greater accuracy, more precision, monitor tremor behavior over time, and provide more non-medicinal options for tremor relief. Even though several products exist to provide lifestyle solutions, none of the products have highly sophisticated smart analytics to detect tremors with precision nor provide the service for tremor characterization and monitoring.

The present invention will address these limitations by empowering those suffering from motion disorders to have a greater deal of control over managing their symptoms and connect with their clinicians in novel ways through highly sophisticated smart analytics.

The invention consists of modules or building blocks that provides solutions to different aspects of care for those with tremor disorders. At present we have defined, designed, and built two modules:
1. Tremor Analysis Module—a non-intrusive wearable unit along with an intelligent software entity which is either local or remote to the unit that will provide the capability of early detection of tremors, monitoring, analysis of tremor behavior and personalized report generation.
2. Non-Intrusive Intervention Module—a module that consists of different types of non-intrusive interventions that aim to dampen tremor activity. These interventions include a TENS unit and a buzzer.

The modules provide unique solutions to the patients, caregivers, and clinicians. Currently we have characterized three broad solutions:
1. Measurement, analysis, and reporting for short-term
2. Monitoring—following a patient's progress over an extended period to assist in treatment planning
3. Interventions—non-invasive and unmedicated therapies to relieve tremors

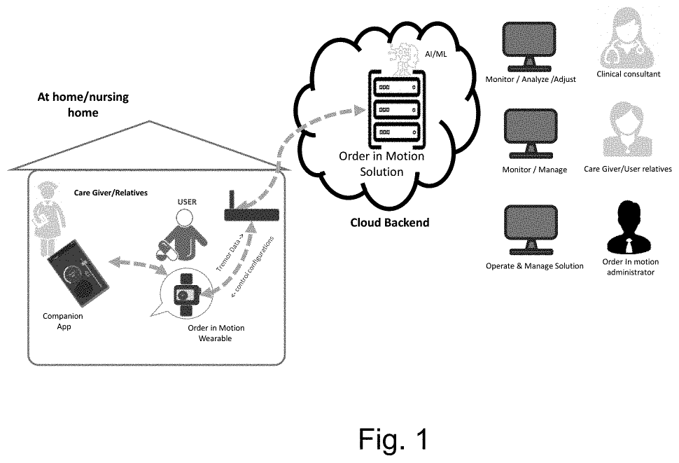

FIG. 1 shows a birds-eye view of how the modules and solutions interface with each other. Once an individual fastens the wearable unit (Tremor Analysis Module) around his/her limb (wrist, ankle) and turns it on, the movements of the limbs are recorded. This recording can be viewed on a device that supports a standard web browser in real or deferred time. Subsequently, this data is stored & analyzed by a remote intelligent software entity, that identifies a distinctive tremor profile for the user. A tremor profile is generated as follows: first the raw data from the forearm motions is processed and the tremor signals are isolated for further analysis. Once, the tremor signals are analyzed, important features of the signals are extracted to give a more quantitative, scientific, and personalized understanding of how a subject's tremors behave over time. These features can be displayed on devices such as a tablet or laptop that supports a standard web browser. Furthermore, a report can be generated summarizing the tremor profile for personal use or discussion with a clinician.

The essence of the present invention is the capture & analysis of tremor behavior resulting from involuntary movements of limbs to identify patterns associated with specific diagnostic groups. These patterns can be a crucial reflection of the underlying pathophysiology that drives tremor disorders, and the sophisticated analytics may be used to help establish a more definitive diagnosis. In addition, by analyzing tremor signals over time, more personalized therapeutic strategies may be provided—thus improving care and daily lifestyle of those saddled by ongoing tremors.

In addition to the wearable unit and associated intelligent software entity, the present invention also contains two interventions that may reduce the impact of tremors. The first intervention is a TENS unit and associated cables and electrodes that provides electrical stimulation which can dampen the intensity of tremors and/or temporarily suspend tremor activity. This element can be manually controlled where the subject or clinician can turn the TENS unit on/off at his/her discretion. Or the TENS intervention can be controlled automatically in response to intensity and duration of tremors. The second intervention is a buzzer.

FIG. 1 shows a concept illustration of how the technology interfaces between users, caregivers and clinicians.

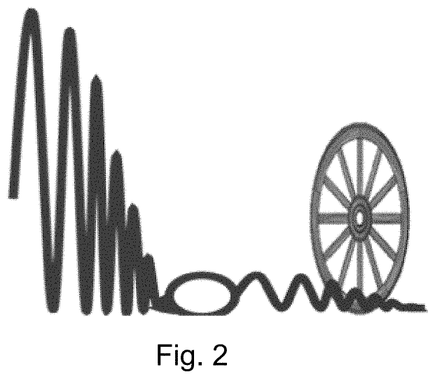

FIG. 2 depicts the Order in Motion company trademark.

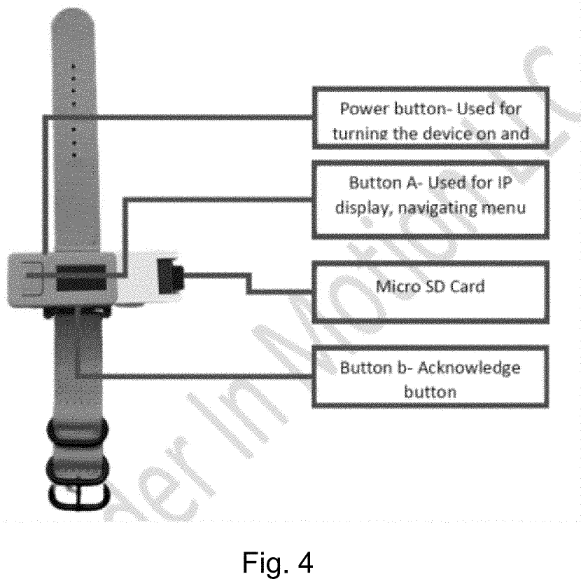

FIG. 4 illustrates the contents of the Order in Motion Wrist Kit which includes the wearable unit, TENS unit and associated cables.

FIG. 4 shows an initial wearable unit for the wrist.

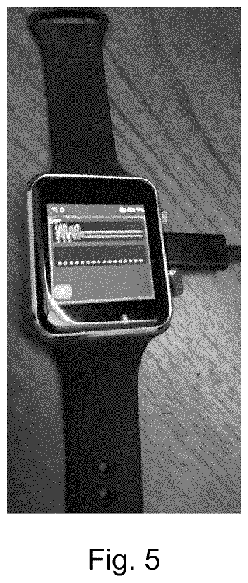

FIG. 5 shows a more technologically advanced wearable unit for the wrist.

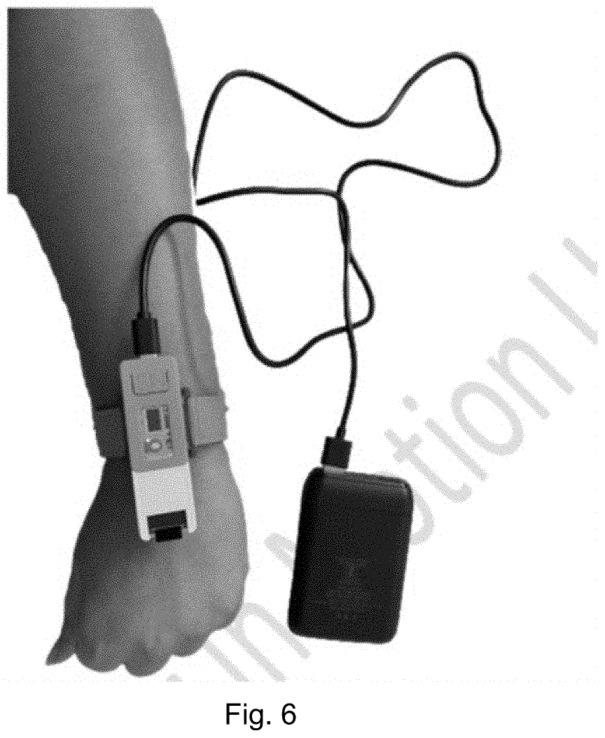

FIG. 6 depicts a subject wearing the unit.

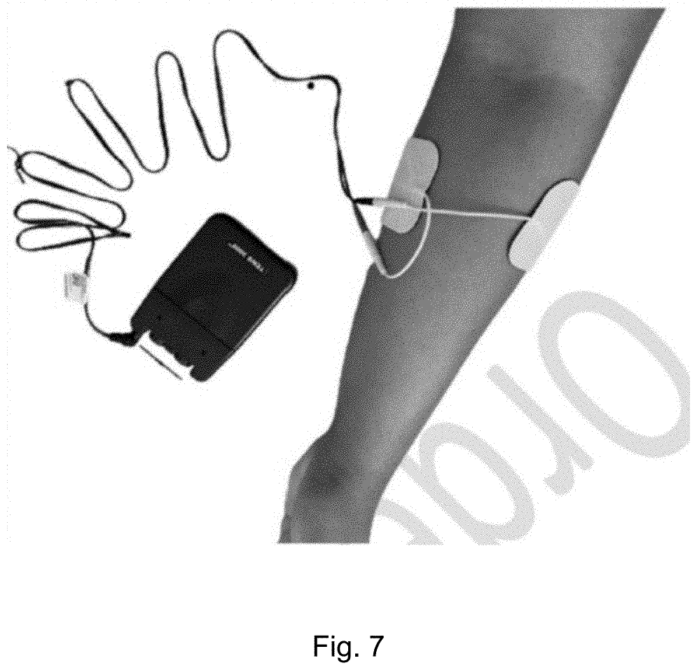

FIG. 7 illustrates a subject connected to the TENS intervention element.

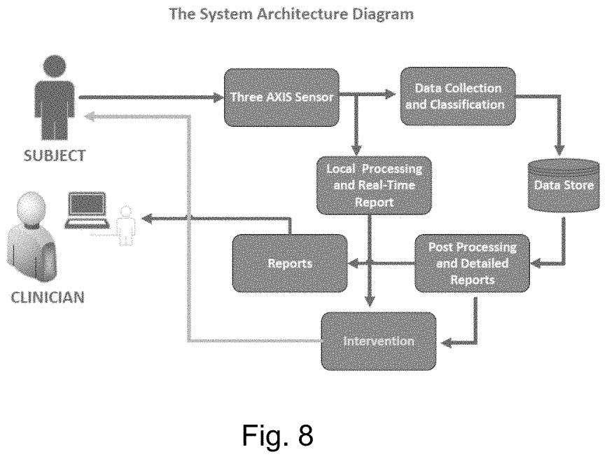

FIG. 8 illustrates the architecture of the present invention.

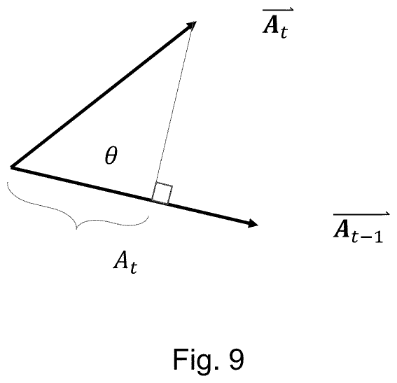

FIG. 9 depicts the vectors for the mathematical algorithm used to combine accelerations from three dimensions into a single net value per time stamp.

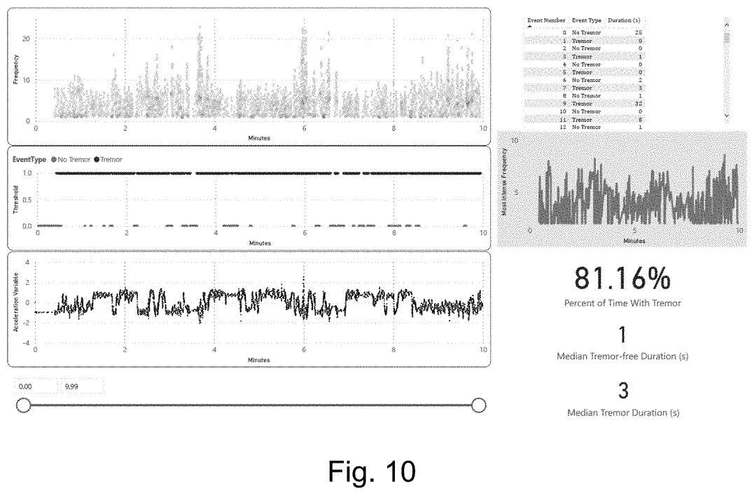

FIG. 10 portrays the home screen of the tremor profile generated by the intelligent software entity.

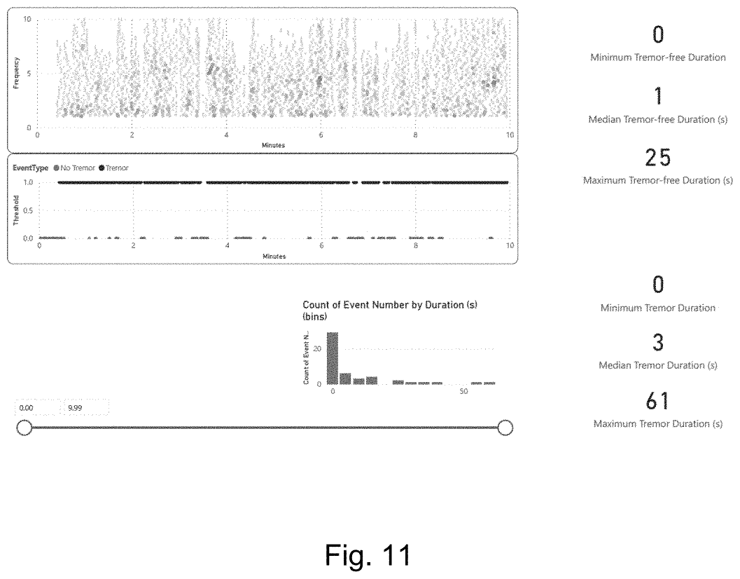

FIG. 11 portrays a secondary screen of the tremor profile generated by the intelligent software entity.

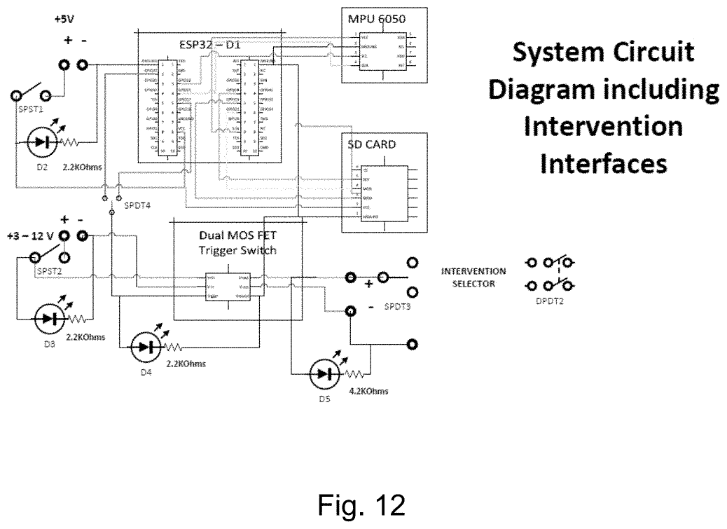

FIG. 12 shows a diagram of the entire circuitry for the present invention.

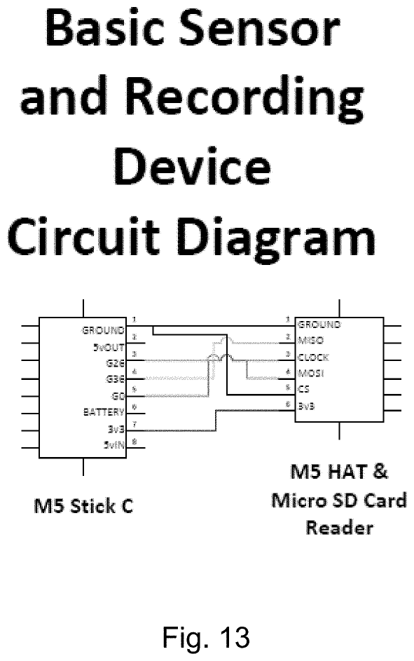

FIG. 13 illustrates the underlying circuitry of the sensor connected to the SD card slot.

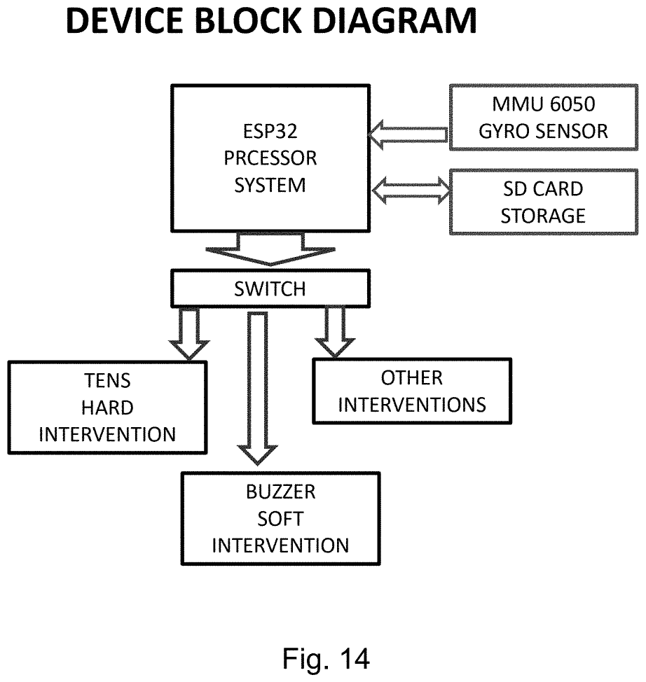

FIG. 14 shows a block diagram of how the major components within the wearable are connected.

### DETAILED DESCRIPTION OF THE INVENTION

The present invention will now be described in more detail. The trademark for the company is illustrated in FIG. 2 and will be stamped onto each individual kit.

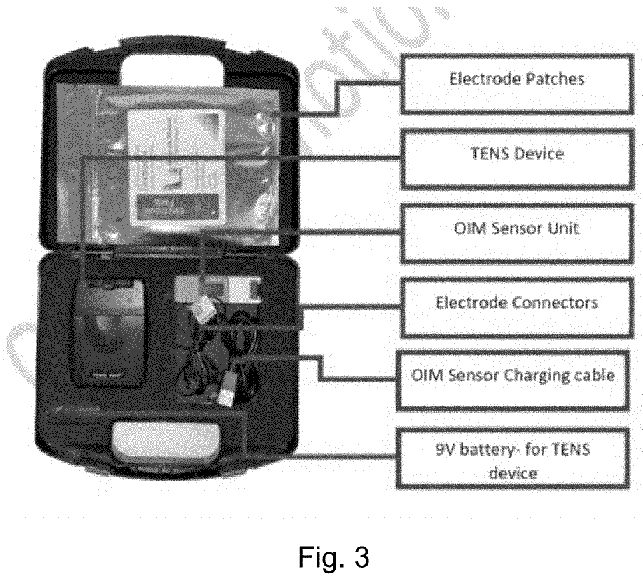

The contents of each kit are depicted in FIG. 3. The kit will include the wearable unit (and charging cable) that detects tremors, collects that data, and relays that information to the software service for further analysis and display. In addition, the kit includes the TENS intervention unit and necessary accessories such as one set of electrodes, one set of electrode connectors, and one 9 Volt battery. Each component of the kit will now be described in more detail. FIG. 3 currently shows only the TENS intervention unit. However, future kits will also include the buzzer intervention unit.

A more detailed and clearer image of the wearable device can be seen in FIG. 4. This figure shows a wearable wrist unit. It includes a Power button, a button for navigating the display, an Acknowledge button, and a slot for an SD card. A more evolved version of this wearable may be seen in FIG. 5. FIG. 6 further shows the unit strapped on snugly on a human wrist, and the device is connected to a phone to view the status and functioning of the wearable unit.

The other major component of the kit is the TENS intervention element (FIG. 7), connected to a human forearm with the electrodes and the corresponding electrode connectors. A standard TENS unit already exists in the market and can be purchased off the shelf. In the present invention, the TENS unit is used in a novel way as an intervention that provides electrical stimulation which may potentially dampen the intensity of involuntary movement and/or temporarily suspend tremor activity. This unit can be manually or automatically controlled. The automatic control mode is NOT part of the standard TENS that can be purchased and is novel to the present invention. When in automatic control mode, the TENS unit turns on and off depending on the onset of tremors, tremor duration, and intensity of tremors. Furthermore, the parameters of this automatic control can be adjusted.

FIG. 8 shows the overarching flow of the present invention, and all the various aspects of the invention previously described will now be collectively explained. A “Subject” is an individual who experiences tremors in one or more of his/her limbs due to some disorder causing involuntary movement. To characterize and monitor this movement, the wearable unit for the wrist can be strapped on that contains the accelerometric sensor. The force of the movement is measured, and the acceleration can be extracted in three dimensions: x, y, and z. The acceleration at each time stamp can be thought of as a vector \vec{A} where \vec{A}=[aₓ, a_{y}, a_{z}]. Next, using a novel mathematical algorithm (Equation 1, Equation 2, and FIG. 9), the acceleration measurements during each time stamp are combined to generate a net acceleration during each time stamp. Thus, the overall acceleration can be viewed over time.

$$cos θ = \frac{\vec{{A}_{t}} \cdot \vec{{A}_{t - 1}}}{‖ \vec{{A}_{t}} ‖ ‖ \vec{{A}_{t - 1}} ‖} \qquad ( Equation 1 )$$

$${A}_{t} = net acceleration value at time t = ‖ \vec{{A}_{t}} ‖ cos θ = \frac{\vec{{A}_{t}} \cdot \vec{{A}_{t - 1}}}{‖ \vec{{A}_{t - 1}} ‖} \qquad ( Equation 2 )$$

In the next stage of processing, the data is put through a signal processing pipeline (part of the intelligent entity) which extracts the involuntary movement signals using a Hanning filter and characterizes interesting aspects of those signals that can be viewed in the tremor profile generated by the intelligent entity. This data can be analyzed over real-time whose results can be seen on a web browser of devices like phone, tablet, or computer. Moreover, this real-time data can be used to help configure & control the TENS intervention, so that the TENS can provide electrical stimulation as needed in response to the nature of the involuntary movements. The raw data will also be stored for more detailed analysis, and reports can be generated into tremor profiles that can be used for clinical purposes such as diagnosis and/or treatment plan adjustment and for various research applications. The tremor profile will next be described.

A tremor profile will have a main page and additional pages for further exploration. FIG. 10 shows the main page generated through the intelligent entity. This main page will graphically show the following: overall measure (acceleration) of the involuntary movement over time, the start/stop of when tremors occur over time, and the intensities (heat map or spectrograph) of the prevalent frequencies of the tremors (including any harmonics that may exist) over time. In addition, the number of tremor events can be viewed during the time recorded, the percent time with tremor, the median length of a tremor event and the median length of tremor-free duration. Please note that a “tremor event” is defined as a single period of non-stop involuntary movement. “Tremor-free duration” is defined as the amount of time between tremor events.

FIG. 11 illustrates how an additional page for further exploration may look. It will include some of the graphical displays from the main page, and additional information such as the number of tremor events binned by duration, the range of the length of a tremor event (minimum and maximum), the range of the length of tremor-free duration (minimum and maximum).

There are multiple versions of the hardware of the wearable units, which have some variations between each other, but essentially perform the similar functions in terms of utility at the systems level (user level). One version of this hardware can be seen in FIG. 12 which shows the main circuitry components for the invention and the connections for a supplemental intervention (TENS). A multi-dimensional motion sensor unit, and other embodiments of this module exist in other hardware versions. The SD micro card is the local non-volatile memory card that can store tremor data along with other information/data used for configuration and functioning of the wearable unit. The ESP32 module is the main processing unit. An alternative embodiment of the ESP32 module is shown in FIG. 13, labelled as the M5 Stick C. FIG. 14 shows how the ESP32 module is connected to the gyrosensor (for detecting acceleration), the SD memory card, and the switch for attachable tremor relieving interventions.

### REFERENCES

1. Louis E D, Ottman R. How many people in the USA have essential tremor? Deriving a population estimate based on epidemiological data, Tremor Other Hyperkinet Mov (N Y), 2014 Aug 14;4:259.
2. Parkinson's Foundation. Statistics; [cited 2021 Feb. 14]; Available from: http://www.parkinson.org/Understanding-Parkinsons/Statistics
3. Caregiver Products. The Wright Stuff. Weighted Hand Writing Glove; [cited 2021 Feb. 14]; Available from: https://www.caregiverproducts.com/weighted-hand-writing-glove.html.
4. Five Microns. Tremelo; [cited 2021 Feb. 14]; Available from:https://fivemicrons.com/.
5. Steadiwear. Steadiwear Inc. The Steadi-One|All-in-one Assistive Glove for Essential Hand Tremor Relief; [cited 2021 Feb. 14]; Available from: https://steadiwear.com/products/parkinson-glove.
6. Readi Steadi. Readi Steadi Anti-Tremor Orthotic Glove System; [cited 2021 Feb. 14]; Available from: https://www.readi-steadi.com
7. Schrag Munchau A, Bhatia K P, Quinn N P, Marsden C D. Essential tremor: an over diagnosed condition? J Neurol. 2000;247:955-959.
8. Jain S, Lo S E, Louis E D. Common misdiagnosis of a common neurological disorder: how are we misdiagnosing essential tremor? Arch Neurol. 2006;63:1100-1104.
9. Gavidia M. Poll Finds 1 in 4 People with Parkinson Disease Misdiagnosed. American Journal of Managed Care. 2020—[cited 2021 Feb. 14]. Available from: https://www.ajmc.com/view/poll-finds-1-in-4-people-with-parkinson-disease-misdiagnosed.

What is claimed is:

1. A wearable device that can be worn on the wrists (and other limbs) of individuals that exhibit tremors or involuntary movement, comprising:
a wristband;
a processing unit that includes various circuitry and a gyroscope to measure accelerations in three dimensions;
a power button to turn on/off the sensor unit;
a button to navigate the display of the sensor unit;
a button to acknowledge;
a micro-SD card as a non-volatile memory card;
a mathematical algorithm that analyzes gyroscope three-dimensional acceleration measurements into a single net acceleration;
a signal processing pipeline that filters the acceleration measurements into separate involuntary movement signals (tremors) and voluntary movement signals;
a software program that analyzes tremors and provides a profile of the tremor to identify dominant frequencies that are present, presence of harmonics, percent time with tremors, median tremor duration (including range of tremor duration), and median time between tremors (including range of time between tremors);
a switch that interfaces the processing unit with non-intrusive tremor relieving interventions;
a TENS unit;
a buzzer unit.

2. A TENS unit that is used as an intervention to dampen tremor activity and can automatically be controlled in response to the onset of tremors.

3. A buzzer unit that is used as an intervention to dampen tremor activity.
